(19) United States
(12) Patent Application Publication
(10) Pub. No.: US 2022/0202489 A1
(43) Pub. Date: Jun. 30, 2022

(54) System And Method For Magnetically Mediated Plasma Treatment Of Cancer With Enhanced Selectivity

(71) Applicant: The George Washington University, Washington, DC (US)

(72) Inventors: Michael Keidar, Baltimore, MD (US); Xiaoqian Cheng, McLean, VA (US); Alexey Shashurin, West Lafayette, VA (US); Jonathan Sherman, Potomac, MD (US)

(21) Appl. No.: 17/647,118

(22) Filed: Jan. 5, 2022

Related U.S. Application Data
(63) Continuation of application No. 15/741,183, filed on Dec. 29, 2017, now abandoned.
(60) Provisional application No. 62/187,500, filed on Jul. 1, 2015.

(51) Int. Cl. A61B 18/18; A61N 2/00; A61B 18/04; A61K 48/00; A61K 47/54; A61K 47/69; A61N 2/06

(52) U.S. Cl. CPC A61B 18/18; A61N 2/002; A61B 18/042; B82Y 5/00; A61K 47/555; A61K 47/6925; A61N 2/06; A61K 48/00

(57) ABSTRACT

A system and method of treating an area having cancerous cells. The system includes a plasma device to generate a plasma jet directed at the area having cancerous cells. A magnetic field generator generates a magnetic field directed at the area having cancerous cells. A controller is coupled to the plasma device and the magnetic field generator to control the plasma jet generated by the plasma device and control the magnetic field generated by the magnetic field generator.

### PRIORITY

The present application claims priority to U.S. Provisional Application No. 62/187,500, filed on Jul. 1, 2015, which is hereby incorporated by reference in its entirety.

### GOVERNMENT LICENSE RIGHTS

This invention was made with U.S. government support under Grant No. 1465061 awarded by the National Science Foundation. The Government has certain rights in this invention.

### COPYRIGHT

A portion of the disclosure of this patent document contains material that is subject to copyright protection. The copyright owner has no objection to the facsimile reproduction by anyone of the patent disclosure, as it appears in the Patent and Trademark Office patent files or records, but otherwise reserves all copyright rights whatsoever.

### TECHNICAL FIELD

The present invention relates generally to treatment of cancer cells and specifically use of cold atmospheric plasma in combination with magnetic field generation to target cancer cells.

### BACKGROUND

Cancer is a well-known health issue. There is a large amount of research geared toward effective treatment of cancer. One area of the research has been directed toward methods of eradicating cancerous cells. Many known methods are problematic because although they result in effective eradiation of cancer cells they also kill healthy cells.

It has been reported since the late 1970s that magnetic fields appear to have a strong effect on biological systems. Research of an electromagnetic field effect on biological systems advanced after Wertheimer and Leeper [1979] found that the likelihood of developing leukemia in children increased as they were present in 60 Hz electromagnetic fields. As the research progressed, it appeared as though the vibrational energy levels in the ion-protein complex were pumping into the system, which was creating parametric resonance. This occurs when the atoms shake slightly. This “shaking” is an anomaly that can change ion flux through the cell membrane or cell mobility. It has been shown that extremely low frequency (ELF) magnetic fields influence physiological processes such as plasma membrane structure modification and the initiation of the signal cascade pathways interference in different organisms. Cell membrane morphology modification by ELF was again reaffirmed by Ikehara et al. [2003], who found that exposure to the ELF magnetic field has reversible effects on the N—H inplane bending and C—N stretching vibrations of peptide linkages, and changes the secondary structures of α-helix and β-sheet in cell membrane proteins.

In the past few decades, cold atmospheric plasma (CAP) has been widely used in various fields such as material processing, bacterial inactivation, wound healing, cut coagulation, cancer therapy, and viral destruction. The temperature of heavy species in CAP is usually close to room temperature, allowing its application to living tissue treatment. Although plasma can selectively kill cancer cells, long time exposure can still damage the normal cells around the tumor.

As proved by numerous studies, cold atmospheric plasma can kill various kinds of cancer but enhancing the efficiency of cold atmospheric plasma on cancer therapy has not been widely studied.

Cells are also being tested in order to examine how static magnetic fields (SMFs) affect apoptosis. Based on the findings of Fanelli et al. [1999], SMFs (0.6-6 mT) exert a strong and reproducible effect of reducing U937 and CEM (normal cell lines) apoptosis. This effect is mediated by the ability of magnetic fields to increase Ca²⁺ influx since its inhibition abrogated the antiapoptotic effect of the magnetic field. On the other hand, Raylman et al. [1996] showed the growth of three cancerous cell lines (HTB 63, HTB 77 IP3, and CCL 86) exhibited a significant reduction in viability after lengthy exposures (64 hours) to very high uniform static magnetic fields at 7 T. Potenza et al. [2004] reported that alterations in terms of increased Escherichia coli cell proliferation and changes in gene expression with a long incubation time (up to 50 hours) were induced by static magnetic field.

Thus, there is a need for a combined cold atmospheric plasma and magnetic field based system for eliminating cancer cells. There is a further need for incorporation of a magnetic field to selectively apply the plasma to an infected area. There is a further need for a cold atmospheric plasma based system that preserves healthy cells while eliminating cancer cells.

### SUMMARY

According to one example, a system for treatment of an area having cancerous cells is disclosed. The system includes a plasma device to generate a cold atmospheric plasma jet directed at the area having cancerous cells. A magnet generates a magnetic field directed at the area having cancerous cells. A controller is coupled to the plasma device to control the plasma jet generated by the plasma device.

Another example is a method of eradicating cancerous cells in an area. A gas is ionized to create a cold atmospheric plasma jet. The plasma jet is directed toward the area of cancerous cells. A magnetic field is generated in the area of cancerous cells.

Another example is a system for treatment of an area having cancerous cells. A plasma device generates a cold atmospheric plasma jet directed at the area having cancerous cells. The system includes a particle container containing nanoparticles. A magnet generates a magnetic field to magnetize the nanoparticles. An injector injects the nanoparticles into the area having cancerous cells. A controller is coupled to the plasma device and the magnet to control the plasma jet generated by the plasma device and control the magnetic field generated by the magnet.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1A is a block diagram of an in vivo system for treatment of an area with cancer cells with a plasma jet and a magnetic field;

FIG. 1B is a block diagram of an in vitro system for eradication of cancer cells with a plasma jet and a magnetic field;

FIG. 1C is a block diagram of another in vivo system for treatment of an area having cancer cells with a plasma jet and magnetic nanoparticles;

FIG. 2A is a perspective view of a testing system for plasma injection and magnetic field application to cells;

FIG. 2B is a side view of a sample container in the testing system shown in FIG. 2A;

FIG. 3 is a table of measured magnetic field strengths from the magnet in relation to the cells shown in FIG. 2B;

FIG. 4A is a graph of the viability of cancer cells normalized by a control group in comparison with use of a plasma jet alone and the application of a magnetic field;

FIG. 4B is a graph showing the plasma spectrum for a plasma jet without the introduction of a magnetic field;

FIG. 4C is a graph showing the plasma spectrum for a plasma jet with the application of a magnetic field;

FIG. 5A is a graph of the viability of cancer cells under a control group in comparison with direct and indirect treatment including a plasma jet alone and a combined plasma jet and magnetic field at a first distance;

FIG. 5B is a close up graph of the viability of cancer cells from direct treatment;

FIG. 5C is a close up graph of the viability of cancer cells from indirect treatment;

FIG. 5D is a graph of the viability of cancer cells under a control group in comparison with direct treatment including a plasma jet alone and a combined plasma jet and magnetic field at a second distance;

FIG. 5E is a graph of the viability of cancer cells under a control group in comparison with indirect treatment including a plasma jet alone and a combined plasma jet and magnetic field at a second distance;

FIG. 5F is a close up graph showing the comparison of direct and indirect combined plasma jet and magnetic treatments;

FIG. 6A is a graph of the viability of cells after a magnetic field treatment alone;

FIG. 6B is a graph of the viability of cells after a plasma and magnetic field treatment;

FIG. 7 is a bar graph of hydrogen peroxide intensity in a culture medium treated by a plasma jet alone and a combined plasma jet and magnetic field;

FIG. 8 is a bar graph of reactive oxygen species intensity in a culture medium treated by a plasma jet alone and a combined plasma jet and magnetic field;

FIG. 9 is a bar graph of cell viability after direct and indirect combined plasma and magnetic field treatments;

FIG. 10A shows the viability of certain cancerous and non-cancerous cells after direct treatment by plasma alone and direct treatment with plasma and a magnetic field;

FIG. 10B shows the viability of certain cancerous and non-cancerous cells after indirect treatment by plasma alone and indirect treatment with plasma and a magnetic field; and

FIG. 11 is a table of resonance frequencies corresponding to a selected group of biologically interactive ions for a plasma jet.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

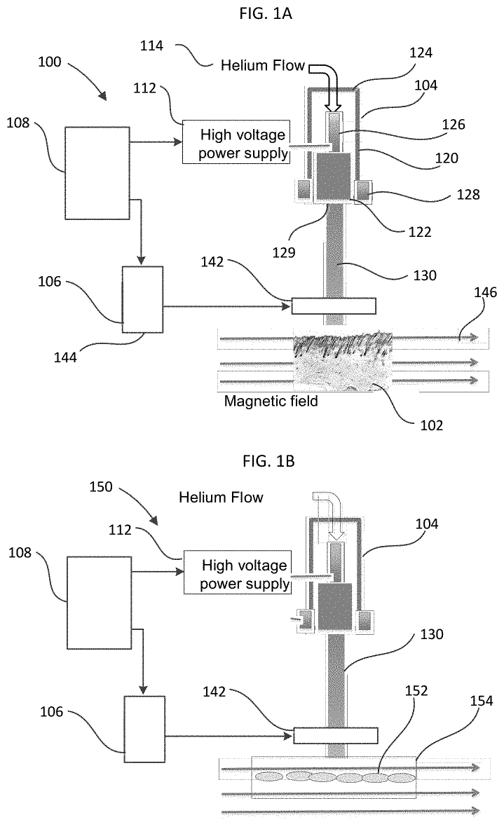

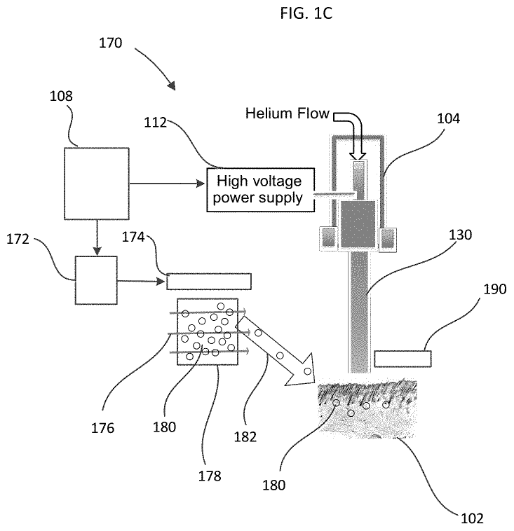

FIG. 1A is a block diagram of an in vivo cancer treatment system 100 that subjects an area of cancerous cells on a patient 102 to a cold atmospheric plasma jet and a magnetic field. The system 100 includes a cold atmospheric plasma emitter device 104 and a magnetic field generator 106. A controller 108 allows a user to control the components and selectively treat cancer cells.

The cold plasma emitter device 104 includes a power supply 112, a gas source 114, and a delivery mechanism 120. In this example, the delivery mechanism 120 is an elongated syringe having a main body 121. The body 121 may be made by glass or a rigid plastic, but also can be made of a flexible material. A proximal end of the body 121 is sealed 124 and an opposite distal end has a discharge area 122. The distal discharge end 122 of the syringe body 121 has a narrowed neck and a distal opening or nozzle 129. A central electrode 126 is located at the center of the body 121 at the interior of the body 121 at the central longitudinal axis of the syringe 120. The central electrode 126 enters the syringe 120 at the sealed proximal end of the body 121 and extends the length of the body 121 to approximately the discharge end 122. A sealing plug 124 (such as rubber) is located over the open end of the syringe 120 to prevent the gas from escaping from the inside of the syringe 120. The electrode 126 is entirely surrounded by insulation except at its distal end, which is exposed and in contact with gas and plasma. The insulation allows the power to be focused at the exposed distal end to lead to the discharge at the end. The central electrode 126 and surrounding insulation, has a proximal end that extends to the outside of the syringe 120 through an opening in the plug 124. The plug opening forms a friction fit with the insulation, so that gas does not escape from the syringe 120. The central electrode 126 is positioned inside the body 121 of the syringe 120, except for the portion of the proximal end of the electrode 126 that extends into and through the plug.

In this manner, the plug opening holds the electrode 126 and insulation in position within the syringe 120, with the distal end of the electrode 126 facing the distal nozzle 129 of the syringe body 121. In addition, an annular outer ring electrode 128 is located about a portion of the narrow neck at the outside of the syringe 120. The electrodes 126 and 128 are high voltage electrodes. The central electrode 126 may be, for instance, a wire, and the insulation can be a ceramic insulation. The high voltage power supply 112 is electrically connected to the electrodes 126 and 128 and provides a high voltage supply to the electrodes 126 and 128 through cables. The controller 108 regulates the voltage and frequency that is applied to the central electrode 126 and the ring electrode 128 and therefore controls the intensity of a plasma jet 130 emitted by the nozzle 129.

The gas source 114 is in gas communication with the delivery device 120 through a supply tube. The supply tube is connected to a port located on the plug 124 of the syringe 120. The supply tube 118 may also be connected to the syringe 120 through an adapter. The gas source 114 can be pressurized, so that gas travels through the supply tube 118 into the inside space of the syringe body 121. A separate gas controller (not shown) may be provided to control the flow rate of the gas in the supply tube 118, or the gas controller may be integrated with the controller 108. The gas then continues through the syringe 120 and exits the syringe 120 through the neck and nozzle 129 at the discharge end 122 as the jet or stream flow 130.

As the gas enters the discharge area 122 and the neck of the syringe 120, the electrodes 126 and 128 excite the gas, thereby ionizing the gas to form a cold plasma jet. In this example, the gas is helium, though other gases such as nitrogen may be used. Thus, as the gas is discharged out of the distal nozzle 129 of the syringe 120, it is a cold plasma jet. The cold plasma jet or stream flow 130 diffuses over time. In accordance with this example, the plasma is provided at a flow rate of 17 liters per minute, with the voltage supply being 5 kV and 30 kHz. At that configuration, the plasma will have a high ionization as it exits the syringe 120. Accordingly, the syringe 120 is preferably placed at a predetermined distance from the target cells of the patient 102 being treated. The syringe 120 allows the plasma to be targeted at desired cancer cells in the skin to selectively eradicate the cancerous cells and reduce tumor size. The syringe 120 may be utilized, for instance, to treat any cancer type that is close to the skin and can be applied without surgery, such as breast, colon, lung, bladder, or oral. With surgery, the system 100 may be applied to any tumor. In this example, the flow rate may be 10-17 liters/min., with a voltage of 2-5 kV and frequency of 30-35 KHz, and a nozzle 129 of 3-5 mm diameter and a distance between the central electrode 126 and the ring electrode 128 of 5-10 mm. The plasma preferably has a density of about 3×10 to 9×10-cm³, such as discussed in “Temporary-resolved measurement of electron density in small atmospheric plasmas,” to Shashurin et al, Applied Physics Letters 96, 171502 (2010), which is hereby incorporated by reference. At the predetermined distance, the plasma will have diffused to a desirable level. However, the intensity of the plasma will continue to decrease as the target area is moved further from the syringe 120, and the plasma will be essentially entirely dissipated at a distance of 5 cm from the syringe 120 in this example. The plasma is well collimated the entire length up to about 5 cm from the syringe 120. The plasma jet stream is discontinuous and represents a series of propagating plasma bundles.

It should be apparent, however, that other suitable settings may be utilized. Preferably, however, the power supply 112 has a voltage from about 2-5 kV with a frequency of about 30 kHz, and the gas has a flow rate of about 2-17 l/min.

The magnetic field generator 106 includes an electromagnet 142 that is coupled to a power regulator 144 to generate a magnetic field 146 around the area of the patient 110. The electromagnet 142 may be moved to focus the magnetic field in the area where the plasma jet stream 130 from the syringe 120 is focused. The strength of the magnetic field 146 may be controlled by the controller 108.

As will be explained below, the plasma jet 130 in combination with the magnetic field 146 in FIG. 1A serves to eradicate cancerous cells but does not have a significant effect on healthy cells surrounding the cancerous cells. The magnetic field 146 and the movement of the syringe 120 in FIG. 1A allows the in vivo treatment to be focused to specific areas on the patient 102 having high concentrations of cancerous cells.

FIG. 1B shows an in vitro system 150 that may be used in conjunction with cells 152 that are contained in a tray 154. The in vitro system 150 may be used for testing treatment parameters or study of cell explant from patient. In this case cells will be treated in order to determine a personalized approach for specific patient. The cell explant may be obtained via a biopsy. Various components in the in vitro system 150 are identical to those of the in vivo system 100 in FIG. 1A and like elements are labeled with identical element numbers. A controller 108 allows control of the plasma device 104 to deliver the plasma jet 130 to the cells 152 in the tray 154. The controller 108 also controls the magnetic field generator 106 to generate the magnetic field 146 through the electro-magnet 142. In this manner, the cells 152 are subjected to exposure to the plasma jet 130 and the magnetic field 146.

FIG. 1C shows a treatment system 170 using magnetic nanoparticles to deliver a magnetic field to the patient 102 in conjunction with a cold atmospheric plasma jet. The cold plasma emitter device 104 is identical to that in FIG. 1A and thus like elements are labeled with like element numbers. As explained above, the plasma emitter device 104 emits a plasma jet 130 directed toward the area of the patient 102 that has a concentration of cancerous cells.

The controller 108 is coupled to a magnetic field generator 172 that controls an electromagnet 174. The electromagnet 174 generates a magnetic field 176 in a particle chamber 178. The particle chamber 178 holds nanoparticles 180 that are magnetized by the magnetic field 174. The magnetized magnetic nanoparticles 180 are delivered to the area of the patient 102 via an injector 182. The magnetic nanoparticles 180 are thus delivered to emit a magnetic field on cancerous cells in conjunction with the plasma jet 130.

The treatment system 170 allows generating the magnetic field in areas with high concentrations of cancerous cells. Thus, the effect of the magnetic field on surrounding areas with normal cells is bypassed. The magnetic nanoparticles 180 target only the cancer cells in the injection region. The magnetic nanoparticles 180 may be further guided by a magnet 190 to a specific location for a more focused treatment. Alternatively, the magnetic nanoparticles 180 may be conjugated with a targeting antibody that may be injected in the area of cancerous cells. Such an arrangement may allow a synergetic effect of plasma, magnetic field, and a drug carried by the magnetic nanoparticles.

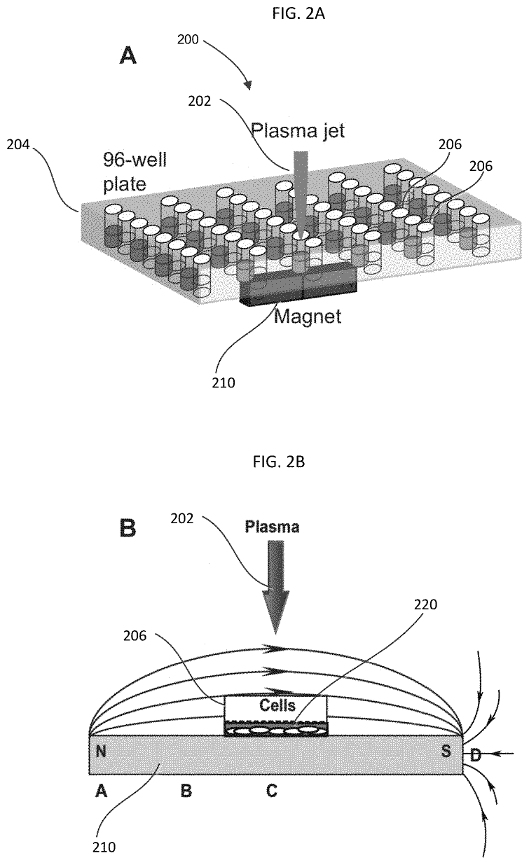

FIG. 2A shows an experimental setup 200 to show the effectiveness of plasma and magnetic field in eradicating cancer cells. The setup 200 includes a cold atmospheric plasma device that produces a plasma jet 202. The plasma jet 202 is directed to a test plate 204 that includes wells 206 that hold the cell samples. A magnet 210 is used to generate a magnetic field. The plasma jet 202 is directed to each of the wells 206 to expose the cells to plasma and the magnet 210 may be used to generate a magnetic field on the wells 206.

FIG. 2B is a close up cross section view of a series of cells 220 that are placed in a well 206 of the plate 204 in FIG. 2A. As shown in FIG. 2B, the magnet 210 generates a magnetic field through the cells 220 when the plasma jet 202 is directed to the cells 220.

The cold plasma device in this example produces the plasma jet 202. In this example, the cold plasma device has a configuration of central powered electrode of 1 mm diameter coating with 2 mm ceramic layer and a grounded outer electrode wrapped around the outside of a 4.5 mm diameter quartz tube. The electrodes are connected to a secondary of high voltage resonant transformer with voltage up to 10 kV and a frequency of 30 kHz. The plasma discharge is driven by alternating current (AC) high voltage. The output voltage is set to 3.16 kV. The feeding gas helium (Airgas, Alexandria, Va.) is set at a flow rate of 4.7 l/min. The distance between the cold atmospheric plasma nozzle emitting the plasma jet 202 and the plate 204 was set to 3.5 cm in this example.

The permanent magnet 210 is used to provide a static magnetic field. The magnetic field strength is measured by a gauss meter (GM08 by Hirst Magnetic Instruments, Falmouth, UK). The magnetic fields at the vertex, quarter, center, and end points (spots A, B, C, and D) on the magnet 210 were tested. In this example, various cancerous and healthy cells were tested as well as different conditions such as without a magnetic field to test the effectiveness of the plasma and magnetic field in eradicating cancer cells. Different areas of the magnet 210 for location of the cells shown as spots A, B, C, and D in FIG. 2B relative to the magnet 210 were also tested.

In this example, human MDA-MB-231 breast cancer cells were used. In order to show the selective effect of plasma, wild type mouse dermal fibroblasts (WTDF) were also tested under the same conditions. The cells were cultured in Dulbecco's Modified Eagle Medium (Life Technologies) supplemented with 10% (v/v) fetal bovine serum (Atlantic Biologicals, Frederick, Md.) and 1% (v/v) Penicillin and Streptomycin (Life Technologies, Grand Island, N.Y.). Cultures were maintained at 37° C. in a humidified incubator containing 5% (v/v) CO2 (Airgas, Alexandria, Va.). Cells were observed under a Nikon Eclipse TS100 inverted microscope (Nikon Instrument, Md.).

The testing involved both direct and indirect treatment using cold atmospheric plasma. The direct treatment involved pre-seeding cells in 96-well plates such as the plate 204. The 96-well plate 204 may be a Costar 96-well plate available from Sigma-Aldrich, St. Louis, Mo. After 24 hours of incubation the culture media in the wells was replaced by 100 μl fresh culture media. The cells were treated directly under the plasma jet from the plasma device alone and in combination with applying the magnetic field. The magnet 210 was stationary at all times during the treatment. The tip of the plasma jet 202 was aligned with the magnet 210 at a desired spot. The 96-well plate 204 was placed on the magnet 210 and the plate 204 was moved from well to well 206 for exposure to the plasma jet. Since the size of the plasma jet is slightly bigger than the area of each well 206, the cells were plated in every other row and every other column to avoid triple or quadric plasma treatment.

The indirect treatment using cold atmospheric plasma involved warming the cell culture media up to 37° C. and adding the media in blank 96-well plates (100 μl per well) and then treating the cell media by plasma with and without the magnetic field. After treatment, the CAP-stimulated media was immediately transferred to affect the cells, which had been pre-cultured in a 96-well plate for 24 hours (the old media was discarded).

In order to compare the cell activity of plasma treatment with and without the application of a magnetic field, cell viability was monitored using the MTT assay (Sigma-Aldrich, St. Louis, Mo.), which is a colorimetric assay for measuring the activity of mitochondria and cellular dehydrogenase enzymes that reduce 3-[4,5-dimethylthiazol-2-yl]-2,5-dyphenyltetrazolium bromide, MTT, to its insoluble formazan, giving a purple color.

The cells were plated at a confluence of 30000 ml⁻¹, and then incubated for one day to ensure a proper cell adherence and stability. Before treatment, cells were replaced with fresh media, and treated with direct or indirect cold atmospheric plasma followed by an additional incubation at 37° C. for 72 hours. After the incubation, 100 μl of MTT solution per well (7 mg Thiazolyl Blue Tetrazolium Blue in 10 ml medium for one 96-well plate) was added into each well. Reactions were maintained for three hours at 37° C. The MTT solution was aspirated and 100 μl of MTT solvent (0.4% (v/ v) HCl in anhydrous isopropanol) was added to each well to dissolve formazan crystals. Reactions were monitored by a Synergy H1 Hybrid Multi-Mode Microplate Reader (BioTek Instruments, Winooski, Vt.) at a wavelength of 570 nm. The entire set of experiments was repeated four times in duplicates.

In this study, the spectra of the plasma jet alone and with the presence of a static magnetic field were measured to detect the difference of reactive species variation in these two experimental conditions. The spectrometer and the detection probe were purchased from Stellar Net of Tampa, Fla. Integration time of the collecting data was set to 100 ms.

5,6-Chloromethyl-29, 79-dichlorodihydrofluorescein diacetate (CM-H2DCFDA) was purchased from Invitrogen for the general intracellular reactive oxygen species (ROS) measurement. The MDA-MB-231 cells were plated in 96 well plates with 100 μl media and treated as required. Two hours after the treatment, 10 μl of 10X CM-H2DCFDA solution in PBS was added in each well to reach the final concentration of 10 nM. 30 minutes later, the intensity of the fluorescence was read by a Synergy H1 Hybrid Multi-Mode Microplate Reader at an excitation wavelength of 492 nm and emission wavelength of 527 nm. The sensitivity of the reader was set to 100.

A hydrogen peroxide (H₂O₂) detection kit from Sigma-Aldrich of St. Louis, Mo. was used. The experiment was performed according to the detailed protocols given on the official website. Cells were plated in black clear-bottom 96 well plates. Immediately after the required treatment, the fluorescence intensity of the H₂O₂ was obtained with a microplate reader (Synergy H1 Hybrid Multi-Mode) at an excitation wavelength of 540 nm and an emission wavelength of 590 nm. Sensitivity of the reader was set to 60.

Results of four repetitions of each experiment were plotted using Microsoft Excel software (2011) as mean ±standard deviation. Student t-test (for comparison between two groups) and one way ANOVA (for comparison between three groups) were used to check the statistical significance (p<0.05).

The application of cold atmospheric plasma and a static magnetic field were integrated using the testing system 200 shown in FIG. 2A. The location of cells on the magnet section determined the effective conditions of the static magnetic field such as direction and strength. Thus, the magnetic fields at the vertex, quarter point, center, and end of the magnet 210 in FIG. 2B were tested (spots A, B, C, and D).

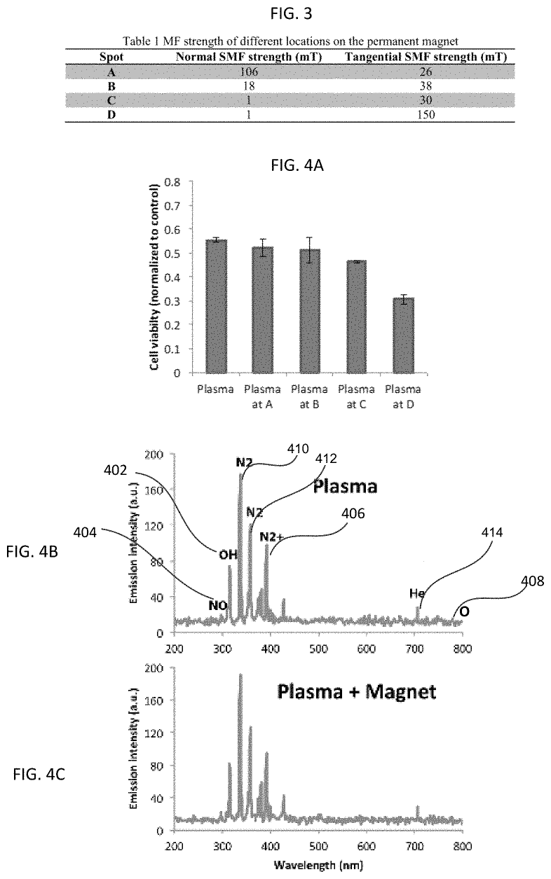

The values of the magnetic field strengths at different spots A, B, C, and D in FIG. 2B are shown in the table in FIG. 3. FIG. 3 shows the measured static magnetic field strengths and the tangential field strengths of the magnetic fields at the spots A, B, C, and D of the magnet 210. As shown in FIG. 3, the normal static magnetic field strengths for spots A, B, C, and D were 106, 18, 1, 1 mT respectively; and the tangential magnetic field strengths for the spots were 26, 38, 30, 150 mT respectively.

U87 cells, the most robust among the four cancer cell lines explained below, were used to test the optimal spot on the magnet 210 having the most significant effect on the cancer cell destruction. FIG. 4A is a graph that shows the cell viability of U87 cells treated by plasma with and without magnetic field at spots A, B, C, and D on the magnet 210 in FIG. 2B. The bars in FIG. 4A represent the cell viability normalized to a control for plasma treatment alone without the magnetic field and the viability with plasma treatment combined with magnetic field application at spots A, B, C, and D in FIG. 2B. U87 cells were plated in the 96-well plate at a density of 3000 cells per well, then treated for 30 seconds with plasma and a static magnetic field (at spots A, B, C, and D) after a 24-hour incubation period. The MTT assay of cells treated by plasma with and without the application of the static magnetic field was performed at a 72-hour time point after treatment to allow the difference to magnify over incubation time. The viability of the experiments repeated three times and were consistent at spots A, B, and C, showing a decreasing viability pattern. While at spot D, however, the cell viability after plasma treatment demonstrated an instability (data not shown). Therefore, spot C is the optimal treatment point that shows the most significant interaction with plasma. The magnetic field strength at spot C is 0.03 T.

The spectra of a normal helium plasma jet alone and with a static magnetic field at Spot C in FIG. 2B was measured. FIG. 4B is a spectra graph of the plasma spectra of the plasma jet alone without the application of the static magnetic field. FIG. 4C is a spectra graph of the plasma spectra of the plasma jet with the application of the magnetic field. FIGS. 4B and 4C show peaks at different wavelengths representing the peaks of emission intensity of different plasma species. An OH peak 402 (309 nm), an NO peak 404 (296 nm), an N₂⁺ peak 406 (391 nm), and an O peak 408 (777 nm) is shown in FIG. 4B. These species are believed to be the key species in the plasma jet to affect the biological system of the cells. Other peaks include two N2 peaks 410 and 412 at different excitations and an He peak 414. As shown in FIGS. 4B and 4C, the identical spectra indicate that the static magnetic field does not affect the generation of the reactive plasma species.

To understand the interaction between the static magnetic field and plasma, cells were isolated from the treatment. Previous studies have demonstrated that the chemical components of plasma-stimulated culture media are modified by the plasma treatment, and this activated media is also capable of inducing the death of cancer cells. The way that cells treated by plasma-activated media, rather than plasma directly, is termed indirect treatment, while the cells treated by plasma jet is termed direct treatment. The indirect treatment offers the possibility of cell isolation from the system of a static magnetic field and plasma.

Cell viability assay was performed to understand the interaction between the static magnetic field, the plasma jet, and the cells. In this study MDA-MB-231 human breast cancer cells were used. In order to show the selective effect of the plasma treatment, wild type mouse dermal fibroblasts (WTDF) cells were also tested under the same conditions. All the cells were also plated at a density of 3000/well and incubated for 24 hours before treatment. The MTT assay was performed at 72 hours after treatment. The viability of cancerous and normal cell lines directly and indirectly treated by helium plasma jet only, a plasma jet with a static magnetic field (SMF), and a plasma jet with a copper board at Spot C on the magnet 210 was assessed.

The cold atmospheric plasma jet, a weakly ionized gas, may be intensified by coupling it to the conductive plate of the magnet 210. Therefore, with magnet 210 being a conductor, the distance between the nozzle of the plasma device and the magnet surface is delicate. If the distance is too far, the intensity of the plasma jet will be significantly reduced so that very little amount of reactive species can reach the media or cells, while if it is too close, the plasma jet will be enhanced at the tip of the jet where it is in contact with the media. The plasma jet coupled to a conducting plate may lead to a much higher amount of the ionized species than the plasma treatment without a magnet, making the data incomparable. Therefore, a non-magnetic ferrite bar of the same material was used as a conductor to replace the magnet 210. The non-magnetic ferrite bar eliminates the factor of the plasma jet enhancement from a conducting plate.

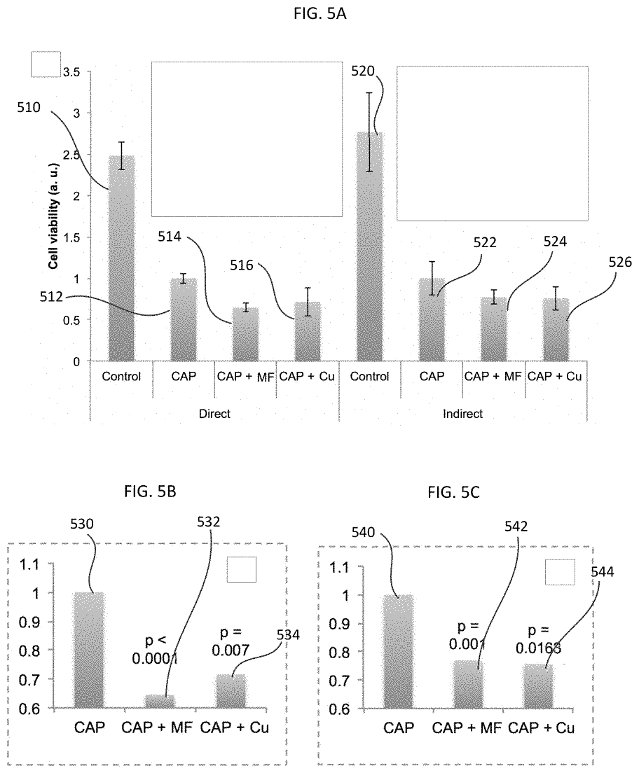

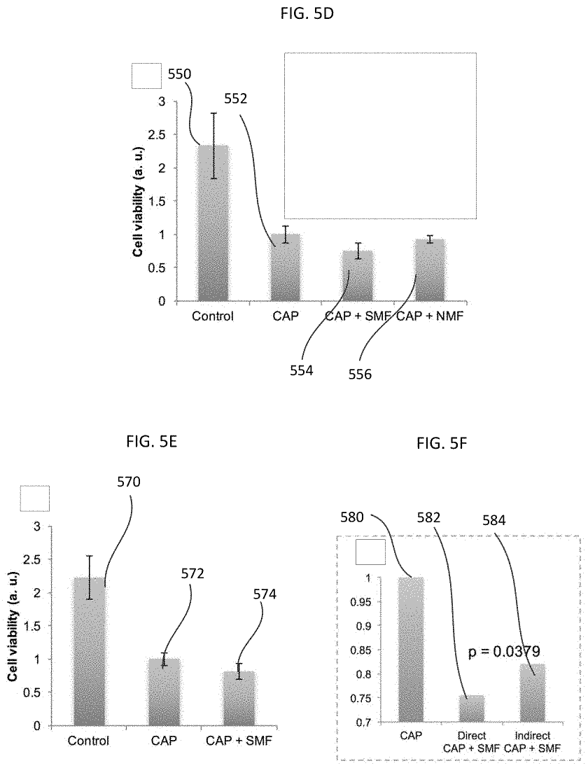

The results of the testing are shown in FIGS. 5A-5C. FIG. 5A is a bar graph of cancerous cell viability for direct and indirect treatment from the testing system 200 in FIG. 2A where the plasma jet is at a distance of 30 mm from the cells. The cancerous cells in this example were MDA-MB-231 breast cancer cells. A bar 510 represents the cell viability in arbitrary units without any treatment. A bar 512 represents the cell viability when the cells are directly treated with cold atmospheric plasma alone. A bar 514 represents the cell viability when the cells are directly treated with cold atmospheric plasma and a magnetic field. A bar 516 represents the cell viability when the cells are directly treated with cold atmospheric plasma using a copper bar in place of the magnet 210 as a conductor. A bar 520 represents the cell viability in arbitrary units without any treatment. A bar 522 represents the cell viability when the cells are indirectly treated with cold atmospheric plasma alone. A bar 524 represents the cell viability when the cells are indirectly treated with cold atmospheric plasma and a magnetic field. A bar 526 represents the cell viability when the cells are indirectly treated with cold atmospheric plasma using a copper bar in place of the magnet 210 as a conductor.

FIG. 5B is a close up graph showing the viability percentage using the direct plasma treatment viability represented by a bar 530. A bar 532 shows that the direct cold atmospheric plasma in combination with the magnetic field has a p value of less than 0.0001 and a bar 534 shows that the cold atmospheric plasma using a copper bar in place of the magnet 210 as a conductor has a p value of 0.007.

FIG. 5C is a close up graph showing the viability percentage using the indirect plasma treatment viability represented by a bar 540. A bar 542 shows that the direct cold atmospheric plasma in combination with the magnetic field has a p value of less than 0.0001 and a bar 544 shows that the cold atmospheric plasma using a copper bar in place of the magnet 210 as a conductor has a p value of 0.0163.

A one-way ANOVA test was performed between the cell viability by direct treatment of cold atmospheric plasma and treatment of cold atmospheric plasma with a static magnetic field and treatment of cold atmospheric plasma alone. The p value of this ANOVA test is 1.798E-11, indicating that there are statistical differences between the three treatments. Thus, a student t-test was performed between the three groups of treatments to determine where the significance lay. As presented in FIG. 5B, the viability of the cells treated directly by cold atmospheric plasma is close to the cells treated by cold atmospheric plasma using a copper bar in place of the magnet 210 as a conductor (p value of the t-test is 0.169 indicating no statistically significant difference). The p value of the t-test between treatment of cold atmospheric plasma alone and cold atmospheric plasma and the magnetic field is 2.84E-07, which confirms the significance.

The same calculation was also performed on the indirect treatments as shown in FIG. 5C. The p value of cold atmospheric plasma alone and of cold atmospheric plasma and a magnetic field is 7.086E-06, suggesting that the significance also exists in indirect treatment.

FIGS. 5D-5F are graphs of cell viability from the above test performed when the plasma jet 202 in FIG. 2B is located at a distance of 35 mm from the cells. FIG. 5D shows a bar 550 represents the cell viability in arbitrary units without any treatment through a direct process. A bar 552 represents the cell viability when the cells are directly treated with cold atmospheric plasma alone. A bar 554 represents the cell viability when the cells are directly treated with cold atmospheric plasma and a magnetic field. A bar 556 represents the cell viability when the cells are directly treated with cold atmospheric plasma using a non-magnetic ferrite bar in place of the magnet 210 as a conductor.

FIG. 5E shows a bar 570 represents the cell viability in arbitrary units without any treatment through an indirect process. A bar 572 represents the cell viability when the cells are indirectly treated with cold atmospheric plasma alone. A bar 574 represents the cell viability when the cells are indirectly treated with cold atmospheric plasma and a magnetic field.

FIG. 5F is a close up graph showing the viability percentage using the direct plasma treatment viability represented by a bar 580. A bar 582 shows the direct cold atmospheric plasma in combination with the magnetic field and a bar 584 showing the indirect cold atmospheric plasma in combination with the magnetic field. FIG. 5F shows the viability of cells treated by plasma with a static magnetic field compared to only plasma treatment in direct and indirect ways. The static magnetic field induced 25% more cell death when cells were treated by plasma directly, and 20% more cell death when cells were treated indirectly. The p value of direct and indirect treatment is 0.0379, suggesting a statistically significant difference.

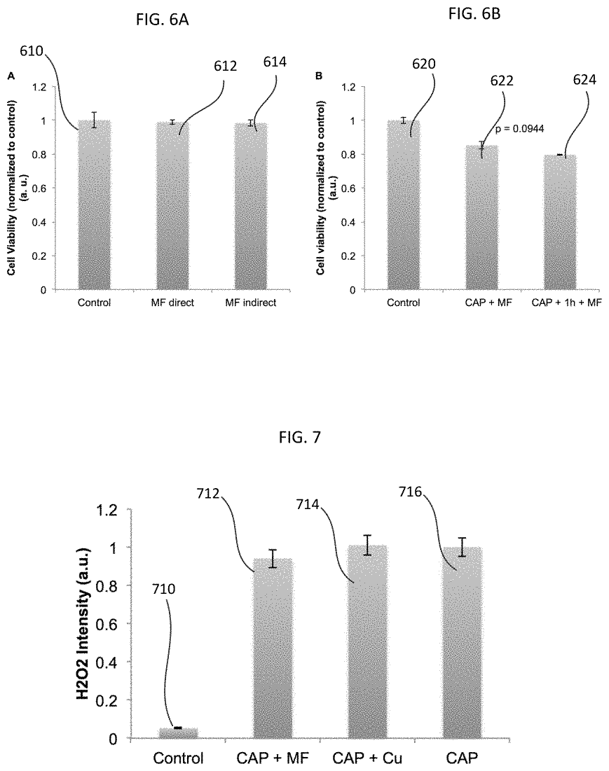

In order to prove that the effect of the magnetic field alone does not have the ability to activate the media or induce cell death, the cell culture media and the cells were placed in the static magnetic field for 30 seconds respectively. The magnetic field treated media was then transferred to infect the pre-plated cells immediately (indirect treatment). Untreated MDA-MB-231 cells were used as a negative control. Cell viability obtained after 72 hours of incubation is shown in FIG. 6A. FIG. 6A is a graph of the cell viability including a bar 610 representing the viability of untreated cells, and bars 612 and 614 representing the viability of cells after direct and indirect treatment of the MDA-MB-231 cells with a magnetic field alone. The viability of magnetic field cells treated directly and the cells treated by static magnetic field activated media is not significantly different from the viability of the untreated cells (p value of ANOVA test is 0.5127), proving that the effect of the 30 seconds of static magnetic field exposure alone can be ruled out from the investigation of the eradication mechanism.

FIG. 6B is a bar graph of the MTT results of the viability of cells that are untreated as represented by a bar 620, cells treated by cold atmospheric plasma and a static magnetic field directly represented as a bar 622 (P+SMF), and cells pre-incubated in the static magnetic field for one hour, then treated with plasma and the static magnetic field directly, represented by a bar 624 (CAP+1h+SMF). The cell viability was also normalized to the untreated group. Although the p value of t-test is 0.0944, meaning the cell viability of one hour pre-incubated cells is not significantly different from cells directly treated by cold atmospheric plasma and magnetic field, the descending trend is consistent (repeated three times). A longer pre-incubation might cause a significant viability decrease.

To further illustrate the static magnetic field does not change the plasma configuration, the generation of H₂O₂ in the culture media treated by plasma alone and plasma with application of the magnetic field was measured (data was normalized to cold atmospheric plasma treatment). FIG. 7 is a bar graph showing the intensity of H₂O₂ in a bar 710 representing a culture media without treatment, a bar 712 representing cold atmospheric plasma with a magnetic field, a bar 714 representing cold atmospheric plasma with a conductor in place of the magnet, and a bar 716 representing cold atmospheric plasma alone. The result of a similar H₂O₂ level was found in the media treated by the cold atmospheric plasma alone and cold atmospheric plasma with a magnetic field. The p value of t-test is 0.199, indicating that the cold atmospheric plasma configuration remains consistent with the application of the static magnetic field.

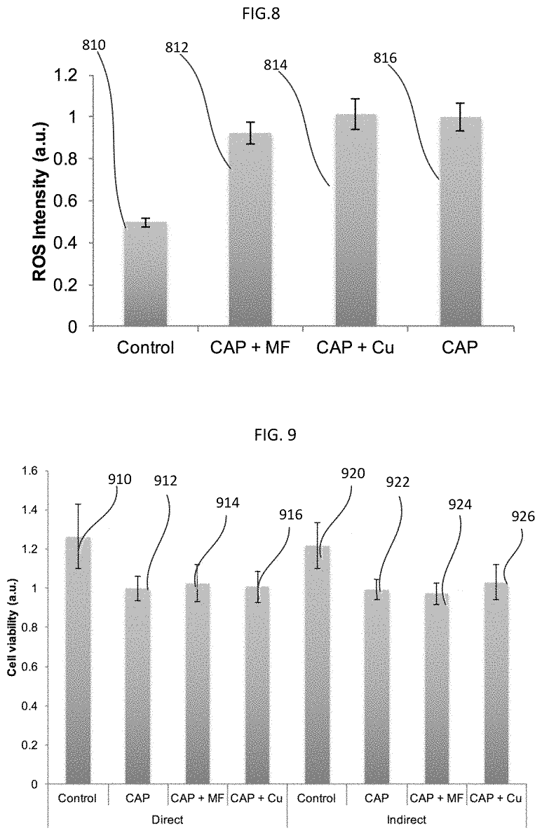

The cold atmospheric plasma treatment may lead to an increased level of free radicals, which has an impact on cellular activity and may explain the decrease of cell viability. Therefore, to determine if the reactive oxygen species pathways are involved in the mechanism of the plasma and magnetic field treatment further decreasing the cell viability, the production of intracellular reactive oxygen species (ROS) was assessed in cells treated by cold atmospheric plasma alone and cold atmospheric plasma with the application of a magnetic field. FIG. 8 is a bar graph showing the intensity of ROS in a bar 810 representing a culture media without treatment, a bar 812 representing cold atmospheric plasma with a magnetic field, a bar 814 representing cold atmospheric plasma with a conductor in place of the magnet, and a bar 816 representing cold atmospheric plasma alone. As shown in FIG. 8, no significant difference in the ROS intensity was observed (p value of t-test is 0.0684), suggesting that ROS pathways are not the dominant eradication mechanism of this study. However, the p value is very close to 0.05, and this could imply that ROS did have a role in this reactions but this effect was diminished by other factors yet unknown.

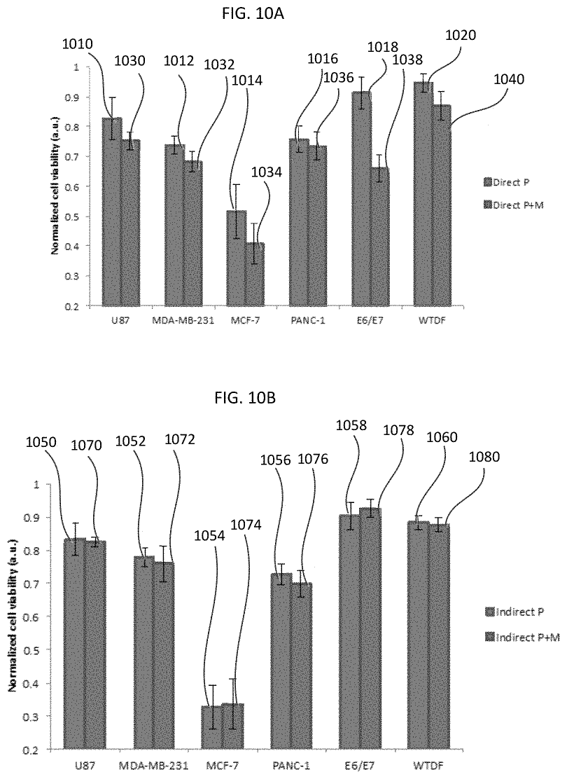

FIG. 9 shows the viability of wild type mouse dermal fibroblasts (WTDF) healthy cells based on direct and indirect plasma based treatment. FIG. 9 includes a bar 910 that represents the viability of WTDF cells with no treatment, a bar 912 representing the viability of WTDF cells with direct plasma treatment alone, a bar 914 representing the viability of WTDF cells with direct plasma and magnetic field treatment, and a bar 916 representing the viability of the WTDF cells with direct plasma treatment using a copper conductor in place of the magnet 210 in FIG. 2B. FIG. 9 also includes a bar 920 that represents the viability of WTDF cells with no indirect treatment, a bar 922 representing the viability of WTDF cells with indirect plasma treatment alone, a bar 914 representing the viability of WTDF cells with indirect plasma and magnetic field treatment, and a bar 916 representing the viability of the WTDF cells with indirect plasma treatment using a copper conductor in place of the magnet 210 in FIG. 2B.

FIG. 9 shows no significant difference between the treatment of plasma alone and plasma and a static magnetic field both directly or indirectly. The results of the WTDF cell viability indicate that plasma treatment leads to the decrease of viability of normal cells around 15%, while the decrease of the viability of MDA-MB-231 breast cancer cells is around 60% as shown in FIG. 5A. Statistically speaking, the odds of cancer cells killed by plasma treatment are 60 to 40, or 6:4=1.5:1, while the odds of normal cells killed by plasma treatment are 15 to 85, or 15:85=0.176:1. Thus the odds ratio is 1.5:0.176=8.5, which means the MDA-MB-231 cells have 8.5 times the odds than the WTDF cells to have been killed by plasma, confirming the selectivity of plasma treatment.

FIGS. 10A and 10B show the results of the viability of four cancer cell lines and two normal cell lines treated by a helium plasma jet only, and a helium plasma jet with a static magnetic field at Spot C shown in FIG. 2B. All the cells were also plated at a density of 3000/well and incubated for 24 hours before treatment. The MTT assay was performed at 24 hours after treatment, and the data plot in FIG. 10A was normalized to cells treated by helium gas (no plasma). FIG. 10A shows bars 1010, 1012, 1014, and 1016 representing the viability of U87, MDA-MB-231, MCF-7, and PANC-1 cancer cells after direct treatment with plasma alone. Bars 1018 and 1020 represent the viability of E6/E7 and WTDF healthy cells after direct treatment with plasma alone. Bars 1030, 1032, 1034, and 1036 represent the viability of U87, MDA-MB-231, MCF-7, and PANC-1 cancer cells after direct treatment with plasma and a magnetic field. Bars 1038 and 1040 represent the viability of E6/E7 and WTDF healthy cells after direct treatment with plasma and a magnetic field. As shown in FIG. 10A, the magnetic field has more or less enhanced the plasma effect on all the cell lines (cancer and normal). The p value of the two treatment conditions for each cell line was proved to be smaller than 0.05, i.e., a statistically significant difference, except the PANC-1 cells. For example, the U87 glioblastoma cells maintained ˜83% viability after plasma treatment, but with the presence of static magnetic field, the viability of cells lowered —8% more. The interaction of the magnetic field and plasma appeared to be the most significant for MCF-7 cells and E6/E7 cells, given that their viability dropped from 55% to 40% and from 90% to 65% with the presence of magnetic field. However, plasma treatment on PANC-1 cells did not benefit from the magnetic field.

The results shown in FIG. 10A are from direct treatment where the cells with culture media were treated under the plasma jet directly. The static magnetic field does not affect the plasma generation as shown by monitoring the spectra of the plasma jet. To further confirm that the static magnetic field interacted with the biological system of cells rather than the plasma jet, the culture media activated by normal plasma jet as well as the jet with magnetic field was used to affect the cells prepared in the 96-well plates in FIG. 2A. This indirect treatment eliminated the role of cells in the magnetic field.

An MTT assay was used again to compare the cell viability of plasma treatment with or without a magnetic field in the way of indirect treatment as shown in FIG. 10B. FIG. 10B shows bars 1050, 1052, 1054, and 1056 representing viability of U87, MDA-MB-231, MCF-7, and PANC-1 cancer cells after indirect treatment with plasma alone. Bars 1058 and 1060 represent the viability of E6/E7 and WTDF healthy cells after indirect treatment with plasma alone. Bars 1070, 1072, 1034, and 1076 represent the viability of U87, MDA-MB-231, MCF-7, and PANC-1 cancer cells after indirect treatment with plasma and a magnetic field. Bars 1078 and 1080 represent the viability of E6/E7 and WTDF healthy cells after indirect treatment with plasma and a magnetic field. The difference between the viability of cells treated in the two conditions is not statistically significant. The above shows that the use of a magnetic field enhances the efficiency of plasma killing rate on cells through the way of interfering with the cell biological system instead of interacting with plasma jet.

The data presented in the above testing shows that plasma alone, and in combination with a static magnetic field, can selectively induce cancerous cell death. The interaction between cells and plasma has been intensively investigated. In terms of mechanism of cancer therapy, the majority favors the theory of reactive oxygen and nitrogen species (ROS and RNS) generated by plasma (extracellular ROS/RNS) and the intracellular ROS signaling and apoptotic pathways they induce. The intracellular ROS generation is promoted by plasma, which could cause cell death by impairing the function of intracellular regulatory factors. Recent studies have emphasized the importance of H₂O₂ formation in the culture media treated by plasma. The H₂O₂ is majorly formed by two ●OH radicals. The toxicity of plasma is highly dependent on H₂O₂, which has a dominant role in the mechanism of cell death. Reactive nitrogen species (RNS) especially NO and peroxynitrite (ONOOH) are also considered important species that lead to cell death. Peroxynitrite formation in the plasma activated media is through the reaction of NO₂⁻ with H₂O₂ and H⁺. ONOOH is a powerful oxidant and nitrating agent that is known to be much more damaging to the cells than NO or superoxide because cells readily remove superoxide and NO to reduce their harmful effects, while fail to neutralize ONOOH.

In the treatment system 100 in FIG. 1 that uses plasma combined with a static magnetic field, it is clear from the spectra that the addition of the external static magnetic field does not alter the plasma chemical composition. H₂O₂ production measurement in the media further confirms the stability. The consistency of plasma jet composition guarantees that the effects observed are associated with magnetic field interaction with cells and reactive species in the media. The p value of the production of intracellular ROS assessed in cells treated by cold atmospheric plasma alone and cold atmospheric plasma combined with a static magnetic field was 0.0684. On one hand, it is greater than 0.05, suggesting that ROS pathways might not be the dominant mechanism. However, with p value close to 0.05, it must be acknowledged that the role of ROS could be diminished by the combination effect of the static magnetic field, cold atmospheric plasma, and cells.

The consumption of H₂O₂ by cells over time has been studied. Each cell line consumed H₂O₂ at different rates. The concentration of H₂O₂ halved when stored in room temperature in comparison to −80° C. after three days. However there has been a lack of investigation in the H₂O₂ decay at different time intervals within one day (0, 30 minutes, 1 hour, 6 hours, and 24 hours). Possible future experiments could help discover more about the role of ROS by treating media with plasma alone and plasma with a magnetic field, then adding H₂O₂ to the cells after different time intervals from 0 to 3 days. Cell viability of each post-delayed addition of media can be measured to support the decay of H₂O₂.

As described above, the culture media activated by a plasma jet alone as well as the plasma jet with a static magnetic field was used to affect the cells prepared in the 96-well plates. The indirect treatment isolates cells from the system of the magnetic field and the plasma, showing the interaction is solely between the magnetic field and cells. The results of indirect treatment experiments above have shown that the cold atmospheric plasma and magnetic field activated media can also increase the cell death rate comparing to the plasma activated media alone. However, the cell death rate of indirect treatment is statistically significantly lower than that of direct treatment, suggesting that the mechanism of cells killed by plasma in a static magnetic field could be an outcome of two separate reactions: the magnetic field with cells and the magnetic field with the plasma-activated media.

Previous studies have shown both static and extremely low frequency magnetic fields can interact with biological systems. The possible mechanism of this is the calcium and potassium ions specifically activated by a magnetic field to enhance their transport through membrane ion channels, thereby altering signaling mechanisms and cellular function. While others have demonstrated that prolonged exposure in the static magnetic field may inhibit human cancer cell growth and increase normal cell survival, in the above described tests, a 30-second static magnetic field treatment alone does not induce cell death. Thus the role of static magnetic field effect on the cells may be ruled out of the exploration of mechanism because the cells were not incubated with a static magnetic field for a long period of time as the above studies did.

Cancer cells and normal cells differ in their cell-cell communication, characteristic cell death, repair mechanisms, or other cellular activities. As normal cells, WTDF cells were studied to demonstrate the selectivity of plasma treatment. The breast cancer cells have an 8.5 times higher odds to be killed by plasma treatment than the WTDF cells. To insure plasma and the magnetic field treatment only affect the viability of cancer cells, plasma can be assembled with an endoscope, targeting only an area with a tumor.

Finally, in the system of cells, plasma, and magnetic fields, as discussed above, plasma will generate extracellular ROS and RNS species in media, such as ●OH, H₂O₂, O₂⁻, NO₂⁻, NO₃⁻, and ONOOH. Plasma-produced ROS (or their reaction products) in media then can either diffuse through the plasma membrane or react with the plasma membrane to produce intracellular ROS. Once ROS enter cells, they can damage intracellular components, or promote or inhibit intracellular signaling pathways. Therefore, one possible way to explore the mechanism of plasma and magnetic field synergy is to monitor the intracellular ROS production. However, the matching ROS level in the cells treated by plasma alone and plasma with a magnetic field leads to a second possibility. These radicals or ions could also be activated by the static magnetic field so that their reaction rate with cells is enhanced. This phenomenon is termed parametric resonance, a phenomenon observed in atomic spectroscopy. This model focuses on the magnetic effect in molecules instead of the ion channel, as in original ion cyclotron resonance hypothesis of Liboff.

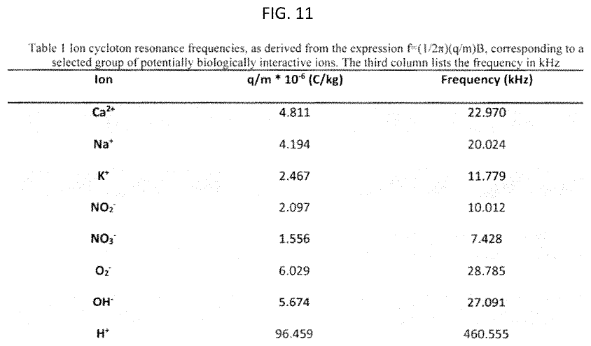

FIG. 11 is a table of a list of ion cyclotron resonances for various biological ions and molecules, including intracellular and extracellular species. As shown in FIG. 11, each ion is selected as a potentially biologically interactive ion. The second column of the table lists the ratio of charge to mass value for each selected ion. The cyclotron resonance frequency is listed in the third column of the table in FIG. 11. The frequency of the key ions in the membrane, Ca²⁺ and Na⁺, as well as OH⁻ and O₂⁻, the predecessors of H₂O₂, all have a frequency that is close to the plasma discharge frequency, which is ˜20-30 kHz. Both the direct and indirect plasma with magnetic field treatment result in significantly lower cell viability and the direct treatment can induce cell death slightly more than indirect treatment. This can be explained as the species in plasma-treated media have cyclotron frequency close to the plasma discharge frequency in a static magnetic field of 30 mT. When cells are directly exposed in the static magnetic field the ion flux through membrane channel might also have a resonance with the plasma discharge frequency.

Combining cold atmospheric plasma and a static magnetic field achieves an enhanced killing effect on cancer cells. In the system of plasma, cells, and magnetic fields, the magnetic field enhances the efficiency of plasma on cancer therapy through interfering with the cell biological system and reactive species instead of interacting with plasma jet. In addition, the magnetic field may be used to guide the plasma-cell interaction region. As such it has promise to enhance selectivity of the region exposed to the treatment.

Each of these embodiments and obvious variations thereof is contemplated as falling within the spirit and scope of the claimed invention, which is set forth in the following claims.

1. A system for treatment of an area having cancerous cells, comprising:
a plasma device to generate a cold atmospheric plasma jet directed at the area having cancerous cells;
a magnet to generate a magnetic field directed at the area having cancerous cells; and
a controller coupled to the plasma device to control the plasma jet generated by the plasma device.

2-20. (canceled)
